(19) United States
(12) Patent Application Publication
(10) Pub. No.: US 2011/0295287 A1
(43) Pub. Date: Dec. 1, 2011

(54) ACTUATION SYSTEM FOR AN ANASTOMOSIS DEVICE

(75) Inventors: Kory P. Hamel, Bloomington, MN (US); Vincent G. Copa, Minnetonka, MN (US); Robert L. Rykhus, JR., Edina, MN (US)

(73) Assignee: AMS RESEARCH CORPORATION, Minnetonka, MN (US)

(21) Appl. No.: 13/142,952

(22) PCT Filed: Dec. 30, 2009
(86) PCT No.: PCT/US09/69826

§ 371 Date: Aug. 8, 2011

Related U.S. Application Data
(60) Provisional application No. 61/141,280, filed on Dec. 30, 2008.

(51) Int. Cl. A61B 17/11

(52) U.S. Cl. 606/153

(57) ABSTRACT

An anastomosis device including an actuation system adapted to control the deployment of the anastomosis device. The anastomosis device includes a watertight barrier for preventing bodily fluids from traversing an actuation wire lumen containing a plurality of actuation wires. The anastomosis device can include a lockout system on a mechanical actuator that prevents disengagement of a grasping assembly until healing from an anastomosis procedure is complete and the anastomosis device is removed.

### PRIORITY CLAIM

The present application claims priority to U.S. Provisional Application Ser. No. 61/141,280, filed Dec. 30, 2008, and entitled “ANASTOMOSIS DEVICE,” which is herein incorporated by reference in its entirety.

### FIELD OF THE DISCLOSURE

The present invention is generally directed to an anastomosis device for performing anastomosis procedures. More specifically, the present invention is directed to a lockable actuation system for an anastomosis device having a watertight barrier preventing bodily fluids from escaping the body through the actuation system.

### BACKGROUND OF THE DISCLOSURE

Anastomosis generally refers surgically rejoining severed biological lumen by linking the ends of a severed biological lumen together and holding the ends together until the lumen is rejoined. Typically, devices for performing anastomosis procedures comprise deployable grasping assemblies, such as, for example, extendible mechanical tines, for gripping the tissue at the ends of the severed biological lumen and holding said ends in place until the lumen is healed and restored. The deployment of the grasping assemblies can be controlled by an actuation system comprising actuation wires linking the anastomosis device to a mechanical actuator outside the patient's body and controllable by operating room personnel. A representative actuation system includes embodiments as disclosed U.S. Patent Publication No. 2006/0206122A1, which is herein incorporated by reference in its entirety.

Typically, the anastomosis device is implanted by mounting the anastomosis device at the tip of the catheter and inserting a portion of the catheter through an incision in the patient's body and into the biological lumen. The catheter can also be inserted through a natural opening in the biological lumen, such as the end of the urethra. The remaining portion of the implantation catheter and the mechanical actuator remain outside the patient's body to allow operating room personnel to control the positioning and deployment of the anastomosis device. Once the anastomosis device is properly positioned inside the patient's body, the actuation system is then used to deploy the grasping assemblies to grip the tissue at the ends of the severed lumen. The implantation catheter is often a double lumen catheter, wherein the actuation wires are disposed within one of the catheter lumen and the mechanical actuator extends from the end of the catheter.

While anastomosis devices are typically removed once the biological lumen has been repaired, the anastomosis device must often remain in place for a substantial period of time until the lumen is restored. Similarly, the implantation catheter and the actuation system also typically remain in place until the lumen is fully healed to simplify the removal procedure. Specifically, the actuation system remains operationally linked to the anastomosis device to avoid the difficult and risky procedure of reattaching the actuation system to the implanted anastomosis device in order to retract the grasping assemblies and remove the anastomosis device. Consequently, a portion of the implantation catheter and the actuation wires remain within the biological lumen until the lumen is restored. Similarly, the mechanical actuator and the end of the catheter extending outside the patient's body also remain in place until the anastomosis device is removed.

While the anastomosis device and the implantation system remain in place during the healing process, bodily fluids can breach the lumen containing the actuation wires and flow through the lumen until leaking out of the patient's body through the mechanical actuator. This leakage can stain clothing or bed linens and contribute to patient aggravation. While the mechanical actuator may be wrapped to minimize the leakage, the wrapping only stems the leakage and may be unable to completely prevent the leakage. As such, there is a need for an apparatus or method to prevent bodily fluids from entering the actuation wire lumen and leaking from the mechanical actuator.

Similarly, because the implantation system remains in place, the implantation system can be used to operate the anastomosis device at any time. As the mechanical actuator remains outside the body and operationally linked to the anastomosis device, patients can inadvertently operate the mechanical actuator to retract the grasping assemblies of the anastomosis device before the biological lumen is fully restored. Retraction of the grasping assemblies can cause the anastomosis device to move within the lumen and damage the lumen or cause the ends of the lumen to re-separate. While control locks are currently available to prevent accidental operation of the mechanical apparatus during the implantation procedure and patient's normal movement, the control locks can be easily disengaged both accidentally and intentionally. As such, there is a need for lockout apparatus or method that prevents any operation of the mechanical actuator until the anastomosis device is removed.

### SUMMARY OF THE DISCLOSURE

A representative embodiment of an anastomosis device of the present invention comprises an actuation system adapted to control the deployment of the anastomosis device. Specifically, the present invention is directed to an actuation system for an anastomosis device having a watertight barrier preventing bodily fluids from breaching the catheter lumen containing the actuation system and leaking from the body. The anastomosis device can further comprise a lockout system preventing disengagement of the control lock until healing is complete and the anastomosis device is removed.

In one aspect, a representative anastomosis device comprises an implantation catheter defining at least one lumen, a plurality of actuation wires, a mechanical actuator and a watertight barrier. The implantation catheter includes an operational end adapted to be inserted into the patient's body as well as a terminal end which remains outside the patient's body. The plurality of actuation wires can be disposed within the at least one lumen defined by the implantation catheter and are operationally linked to the anastomosis device such that mechanical energy applied to the actuation wires causes the anastomosis device to deploy or retract. The watertight barrier is disposed at the operational end of the implantation catheter and is adapted to prevent bodily fluids from entering the lumen containing the actuation wires. The watertight barrier can further comprise watertight ports allowing the actuation wires to move through the watertight barrier without permitting bodily fluids from entering the lumen. The mechanical actuator can be disposed at the terminal end of the implantation catheter and is adapted to mechanically manipulate the actuation wires such that operation of the mechanical actuator controls the deployment of the anastomosis device.

In another aspect, the present invention is directed to a method for preventing leakage of bodily fluids during an anastomosis healing period. The method can comprise providing a catheter defining at least one wire lumen between an anastomosis tip at an operational end and a mechanical actuator at a terminal end, wherein the catheter includes at least one wire lumen between the anastomosis tip and the mechanical actuator. The method can further comprise the step of positioning a watertight gasket in the catheter between the operational end and the terminal end. Finally, the method can comprise sealingly engaging a plurality of actuation wires within the at least one wire lumen with the gasket to create a watertight seal in the at least one wire lumen.

In yet another aspect, an actuation system for an anastomosis device can further comprise a pair of trigger arms and a control lock. The pair of trigger arms can be operationally linked to actuation wires such that pressing the trigger arms together mechanically manipulates the actuation wires to control deployment of the anastomosis device. The trigger arms can further comprise arm apertures adapted to receive a locking member, such as, for example, a standard size suture. The control lock is adapted to be physically inserted between the pair of trigger arms, thereby preventing the trigger arms from being pressed together and locking the anastomosis device. The control lock also comprises a locking aperture that is similar in size and appearance to the arm apertures. The arm apertures and the locking aperture are configured to align when the control lock is inserted between the trigger arms such that a retention aperture is defined. A locking member such as a suture can be tied through the retention apertures to secure the control lock between the trigger arms and prevent actuation of the trigger arms during a healing period.

Another aspect of the present invention involves a method for preventing disengagement of an anastomosis device within a patient during a healing period. Initially, the method comprises providing a catheter having a mechanical actuator including a pair of trigger arms for selectively actuating a grasping assembly at an anastomosis tip. The method further comprises positioning a control lock between the pair of trigger arms, wherein the control lock includes a locking aperture and each trigger arm includes an arm aperture such that alignment of the locking apertures with the arm apertures defines a continuous retention aperture. The method further comprises placing a locking member through the continuous retention aperture to prevent removal of the control lock from between the trigger arms during a healing period.

The above summary of the various representative embodiments of the invention is not intended to describe each illustrated embodiment or every implementation of the invention. Rather, the embodiments are chosen and described so that others skilled in the art can appreciate and understand the principles and practices of the invention. The figures in the detailed description that follow more particularly exemplify these embodiments.

### BRIEF DESCRIPTION OF THE FIGURES

The invention can be completely understood in consideration of the following detailed description of various embodiments of the invention in connection with the accompanying drawings, in which:

FIG. 1 is a plan view of a representative anastomosis device of the present disclosure.

FIG. 2 is a perspective, sectional view of an operational end of the anastomosis device of FIG. 1.

FIG. 3 is a perspective view of a representative embodiment of a mechanical actuator with an engaged control lock according to the present invention.

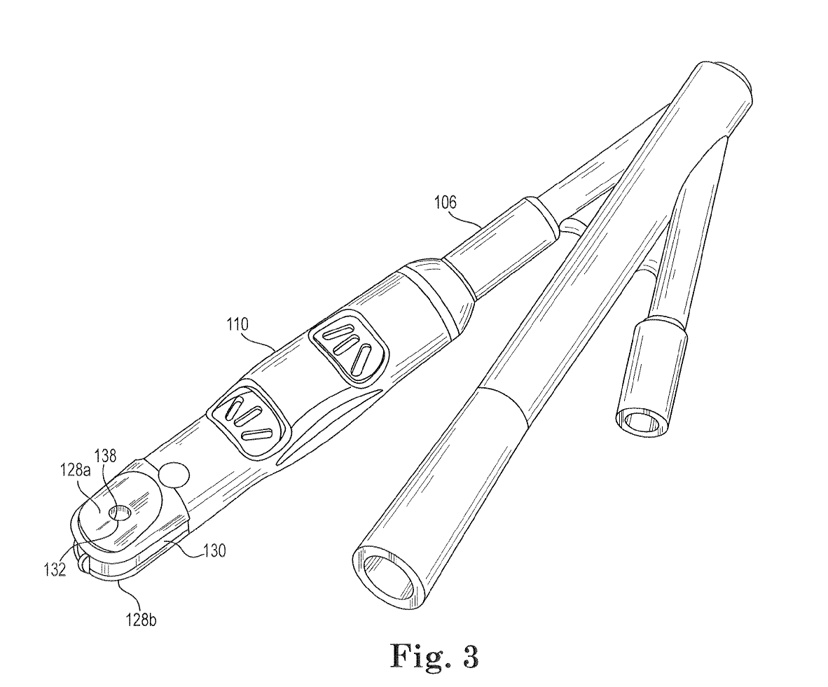

FIG. 4 is a perspective view of the mechanical actuator of FIG. 3 with a disengaged control lock.

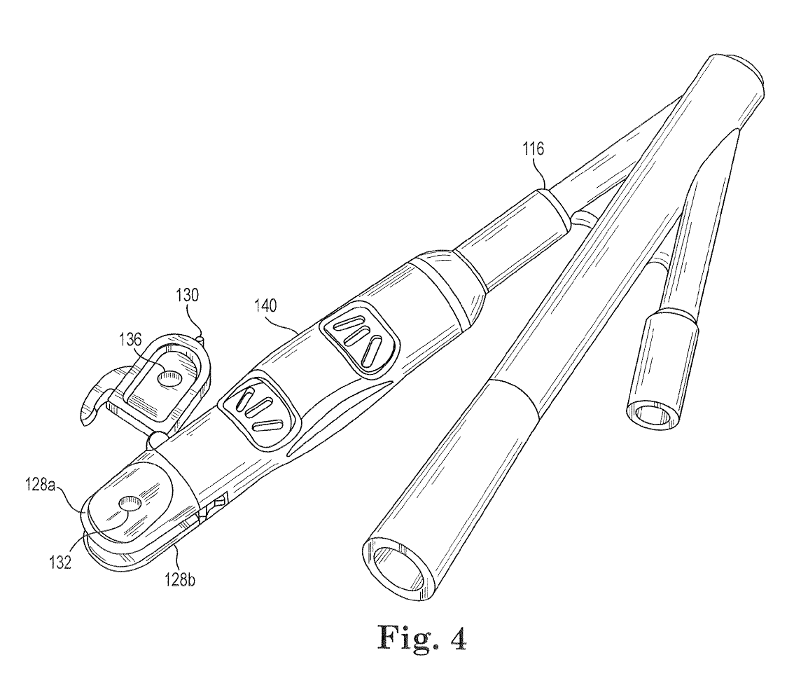

FIG. 5 is a partial section view of a pair of trigger arms on the mechanical actuator.

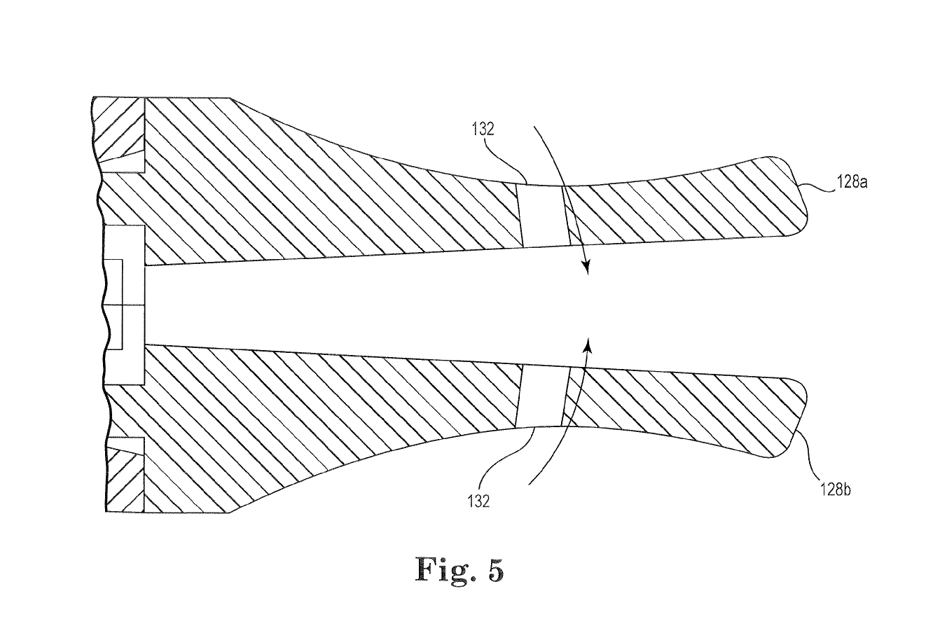

FIG. 6 is a partial section view of the pair of trigger arms and a control lock being retained with a locking member.

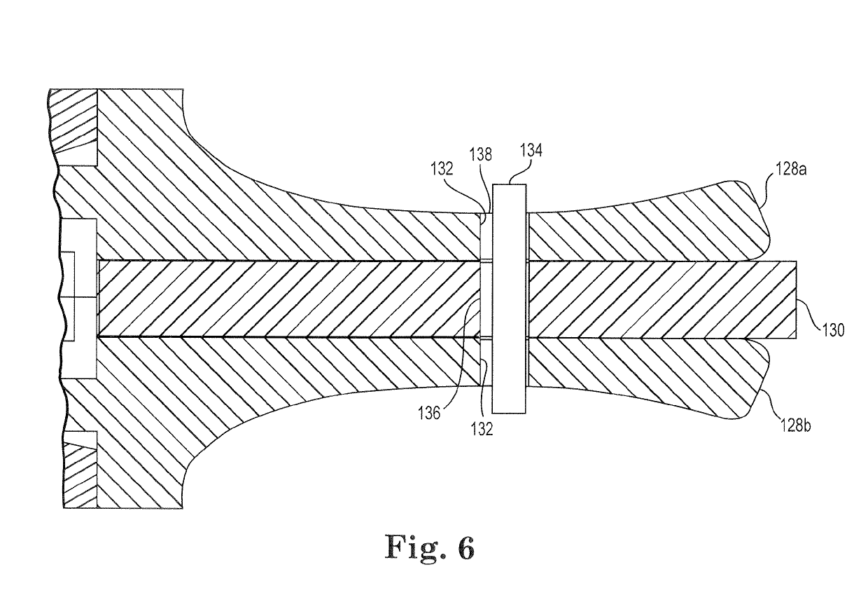

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION OF THE FIGURES

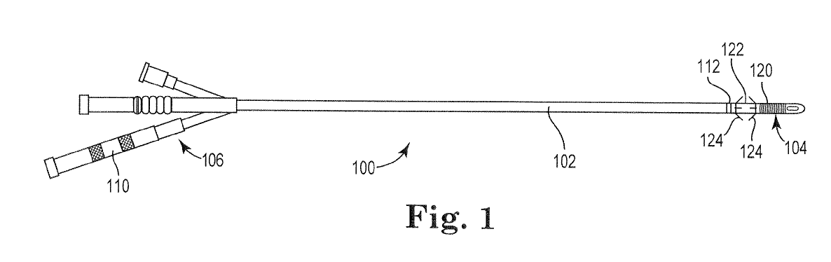

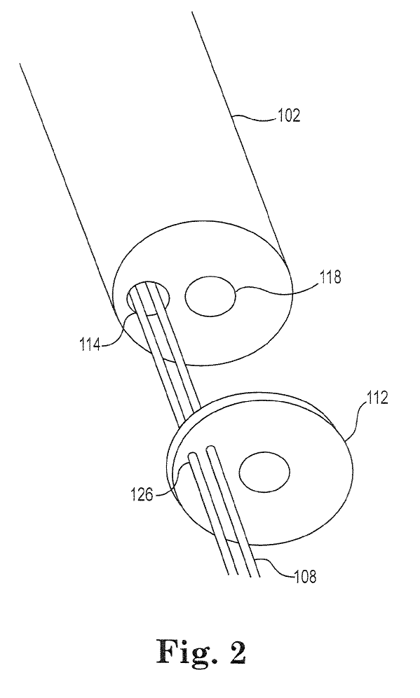

Referring to FIGS. 1 and 2, an anastomosis device 100 of the present invention comprises an implantation catheter 102 having an operational end 104 and a terminal end 106, a plurality of actuation wires 108, a mechanical actuator 110 and a watertight barrier 112. Implantation catheter 102 defines at least one wire lumen 114 extending the length of the implantation catheter 102 and adapted to receive actuation wires 108. Alternatively, implantation catheter 102 further defines at least one general purpose lumen 118 in addition to the wire lumen 114. The general purpose lumen 118 can extend the length of the implantation catheter 110 and be adapted to meet a variety of surgical needs.

Operational end 104 further comprises a catheter tip 120 including a grasping assembly 122 depicted herein as mechanical tines 124. Watertight barrier 112 is generally positioned within the implantation catheter 102 and sealingly engages the actuation wires 108 to prevent bodily fluids from entering the at least one wire lumen 114. Watertight barrier 112 includes watertight ports 126 allowing each actuation wire 108 to pass through the watertight barrier 112 without preventing or otherwise restricting slidable movement of the actuation wires 108 through the watertight barrier 112. In one embodiment, the watertight barrier 112 comprises a gasket made of a soft, flexible material capable of sealing against the actuation wires 108, the at least one wire lumen 114 and the wall of the implantation catheter 102. Representative materials for the watertight barrier 112 can comprise rubber, silicone and other watertight materials.

Actuation wires 108 extend at least from operational end 104 of implantation catheter 102 to the terminal end 106 of implantation catheter 102. Actuation wires 108 are adapted to transfer mechanical energy from one end to the other of the actuation wires 108. The actuation wires 108 in a first position cause the grasping assembly 122 to remain retracted. Moving the actuation wires 108 to a second position causes the grasping assembly 122 to deploy to grip tissue. Actuation wires 108 are operationally linked to the grasping assembly 122 such that mechanically manipulating actuation wires 108 controls the deployment and capture of tissue with the grasping assembly 122. Actuation wires 108 can comprise suitable materials including, for example, metal wires, plastic wires or wires of any other suitable rigid materials.

Mechanical actuator 110 is disposed at terminal end 106 of implantation catheter 102 and is adapted to apply mechanical energy to actuation wires 108 to control the deployment of the grasping assembly 122. Mechanical actuator 110 further comprises a pair of trigger arms 128a, 128b adapted to apply mechanical energy to actuation wires 108 when trigger arms 128a, 128b are pressed together by operating room personnel. Directing trigger arms 128a, 128b together causes the actuation wires 108 to move between a first and second position, which in turn causes grasping assembly 122 to deploy or retract. Mechanical actuator 110 further comprises a control lock 130 that is positionable between trigger arms 128a, 128b to prevent trigger arms 128a, 128b from being pressed together. Trigger arms 128a, 128b each comprise an arm aperture 132 adapted to receive a locking member 134 such as, for example, a standard sized surgical suture. Control lock 130 further comprises a locking aperture 136 sized and configured to receive the locking member 134. When control lock 130 is positioned between the trigger arms 128a, 128b, the arm apertures 132 and locking aperture 136 align to define a continuous retention aperture 138. When the control lock and trigger arms 128a, 128b are aligned to define the continuous retention aperture 138, the locking member 134 can be directed through the continuous retention aperture 138 to prevent the removal of the control lock from between the trigger arms 128a, 128b. In one representative example, the positioning of the locking member 134 through the continuous retention aperture 138 can comprise threading a length of suture through the continuous retention aperture 138 and tying the suture to prevent its removal. Generally, locking member 134 prevents the removal of the control lock 130 from between the trigger arms 128a, 128b such that the grasping assembly 122 is allowed to maintain tissue retention during a healing period following the anastomosis procedure.

Although specific examples have been illustrated and described herein, it will be appreciated by those of ordinary skill in the art that any arrangement calculated to achieve the same purpose could be substituted for the specific examples shown. This application is intended to cover adaptations or variations of the present subject matter. Therefore, it is intended that the invention be defined by the attached claims and their legal equivalents, as well as the following illustrative embodiments.

1. An anastomosis device, comprising:
a catheter having an operational end and a terminal end, the catheter including an anastomosis tip at the operational end and a mechanical actuator at the terminal end, the catheter further defining at least one wire lumen between the anastomosis tip and the mechanical actuator and at least one general purpose lumen fluidly isolated from the at least one wire lumen;
a plurality of actuation wires positioned within the at least one wire lumen, the actuation wires operably connecting the mechanical actuator to a grasping assembly at the anastomosis tip; and
a watertight barrier disposed within the at least one wire lumen between the operational end and the terminal end, the watertight barrier sealing engaging the plurality of actuation wires to prevent transmission of bodily fluids to the terminal end through the at least one wire lumen.

2. The anastomosis device of claim 1, wherein the watertight barrier comprises a gasket positioned adjacent to the anastomosis tip.

3. The anastomosis device of claim 2, wherein the gasket comprises a flexible, watertight material.

4. The anastomosis device of claim 3, wherein the flexible, watertight material comprises rubber or silicone.

5. The anastomosis device of claim 1, wherein the mechanical actuator further comprises a pair of trigger arms operably linked to the plurality of actuation wires, wherein manipulation of the trigger arms causes the grasping assembly to selectively extend and retract from the anastomosis tip.

6. The anastomosis device of claim 5, wherein the mechanical actuator further comprises a control lock adapted for placement between the pair of trigger arms, said control lock preventing the trigger arms from being pressed together when the control lock is positioned between the trigger arms, each trigger arm including a trigger aperture and the control lock including a lock aperture wherein placement of the control lock between the trigger arms aligns the trigger apertures and the lock aperture to define a continuous retention aperture.

7. The anastomosis device of claim 6, wherein a lock member is placed through the continuous retention aperture, said lock member preventing withdrawal of the control lock from between the trigger arms during a healing period.

8. The anastomosis device of claim 7, wherein the lock member comprises a length of suture.

9. An anastomosis device, comprising:
a catheter having an operational end and a terminal end,
an anastomosis tip at the operational end of the catheter,
a mechanical actuator at the terminal end of the catheter, the mechanical actuator including a pair of trigger arms and a control lock, each trigger arm including
a trigger aperture and the control lock including a locking aperture wherein placement of the control lock between the trigger arms defines a continuous retention bore; and
a locking member inserted through the continuous retention bore to prevent removal of the control lock from between the trigger arms during a healing period.

10. The anastomosis device of claim 9, wherein the catheter defines at least one wire lumen between the operational end and the terminal end, the at least one wire lumen including a plurality of actuation wires operably interconnecting a grasping assembly on the anastomosis tip with the trigger arms.

11. The anastomosis device of claim 10, further comprising:
a watertight barrier disposed within the catheter between the operational end and the terminal end, the watertight barrier sealing engaging the plurality of actuation wires to prevent transmission of bodily fluids to the terminal end through the at least one wire lumen.

12. The anastomosis device of claim 11, wherein the watertight barrier comprises a gasket positioned adjacent to the anastomosis tip.

13. The anastomosis device of claim 9, wherein the locking member comprises a length of suture.

14. A method for preventing transmission of bodily fluids during an anastomosis procedure, comprising:
providing a catheter defining at least one wire lumen between an anastomosis tip at an operational end and a mechanical actuator at a terminal end, the catheter defining at least one wire lumen between the anastomosis tip and the mechanical actuator and at least one general purpose lumen fluidly isolated from the at least one wire lumen;
positioning a gasket in the at least one wire lumen between the operational end and the terminal end; and
engaging a plurality of actuation wires within the at least one wire lumen with the gasket to create a watertight seal in the at least one wire lumen.

15. A method for preventing disengagement of an anastomosis device within a patient during a healing period, comprising:
providing a catheter having an anastomosis tip at an operational end and a mechanical actuator at a terminal end, the mechanical actuator including a pair of trigger arms for selectively actuating a grasping assembly at the anastomosis tip;
positioning a control lock between the pair of trigger arms, the control lock including a locking aperture and each trigger arm including an arm aperture, wherein alignment of the locking apertures with the arm apertures defines a continuous retention aperture; and
placing a locking member through the continuous retention aperture to prevent removal of the control lock from between the trigger arms during a healing period.
